(19) United States
(12) Patent Application Publication
(10) Pub. No.: US 2008/0014648 A1
(43) Pub. Date: Jan. 17, 2008

(54) Hazardous Compounds Detector

(76) Inventors: Olivier J. Poncelet, Chalon-Sur-Saone (FR); Francoise M. Thomas, Chalon-Sur-Saone (FR); Gerard A. Friour, Chalon Sur Saone (FR); Jean-Marie Vau, Paris (FR)

Correspondence Address: Carestream Health Inc,, 150 Verona Street, Rochester, NY, 14608, US

(21) Appl. No.: 11/570,501

(22) PCT Filed: Jun. 2, 2005
(86) PCT No.: PCT/EP05/05933

§ 371 Date: Dec. 13, 2006

(30) Foreign Application Priority Data
Jun. 17, 2004 (FR) 0406537

(51) Int. Cl. G01N 21/00; B01J 19/00; G01N 33/00

(52) U.S. Cl. 436/110; 422/50; 436/164

(57) ABSTRACT

The invention relates to a single-use detector of aromatic compounds, with:—a first part (20) comprising a light conversion layer (26), and a photosensitive layer (28) in one piece with the light conversion layer, to be capable of being exposed by exposure light (54) capable of being produced by the light conversion layer in response to excitation light (50), the light conversion layer containing an imprinted polymer precursor (32), capable of combining with a target aromatic compound to form a dye (34) that converts the excitation light into exposure light in a wavelength domain different from that of the exposure light and,—a second part (40) of developer, capable of being applied temporarily to the photosensitive layer (28) of the first part (20) to demonstrate a possible exposure of the photosensitive layer. Application to the detection of explosives.

### FIELD OF THE INVENTION

The present invention relates to a single-use detector for hazardous materials, especially for materials based on aromatic compounds. It also relates to a utilization method for such a detector for detecting the presence of target molecules in an environment.

While the detector can be adapted to various target molecules, a preferred application area of the invention is the detection of explosive molecules of narcotic substances, or the detection of molecules of aromatic compounds, especially nitrated. Applications for the detection of living organisms or pieces of organisms are also possible. This is, for example, spores or pollen.

In particular the invention aims to propose a detector suited to checking baggage or goods in stations or airports.

### BACKGROUND OF THE INVENTION

Various techniques of detection of explosives or hazardous materials are known. One particular illustration is provided by document (1) whose references are given at the end of the description. In this case, this relates to a method of detection of hazardous molecules in the mail.

Document (2), whose references are given at the end of the description, relates to the detection of explosives. For this purpose it proposes using molecular imprinted polymers (MIP). These are polymer molecules capable of combining selectively with the target molecules. MIPs are formed by using the target molecule in a copolymerization reaction, and then removing the target molecule so as to conform the MIP molecule as an “empty print” of the target molecule. Document (2) gives a wide range of monomers that can be used to synthesize MIPs.

Further MIPs have the special feature that their radiation absorption and emission properties are different in their latency state, i.e. before combination with a target molecule, and in the combined state.

Document (2) proposes making use of this property to produce a detector of target molecules. Optical fiber means enable MIP molecules to be coupled to a source and to a light detector. The detection is based on the modification of the fluorescence spectrum.

### SUMMARY OF THE INVENTION

The use of MIPs for the detection of explosives or more generally hazardous molecules is a very attractive solution. However, detection efficiency does not depend uniquely on the MIP molecules. To be efficient, a detector of hazardous molecules must be able to be implemented very easily and very quickly. In addition, it should have a particularly low cost to be able to be implemented not only on a sample of goods, but on all the goods to be carried. Finally, the detector should have an interface enabling a user to have clear and immediate information on the presence or absence of hazardous molecules.

It is an object of the invention to propose a detector of hazardous molecules or compounds, capable of being set up rapidly on goods containers, or being rapidly attached to travelers' baggage.

It is another object to propose such a detector, at low cost, capable of being implemented on a large scale, and with no special knowledge.

Finally, it is an object of the invention to propose a detection method, using the detector.

To achieve these objects, a more precise object of the invention is a single-use detector of target compounds, with:

a first part comprising a light conversion layer, and a photosensitive layer, the photosensitive layer being in one piece with the light conversion layer, to be capable of being exposed by exposure light capable of being produced by the light conversion layer in response to excitation light, and the light conversion layer containing an imprinted polymer precursor, capable of combining with a target compound to form a dye that converts the excitation light into exposure light with wavelength different to that of the excitation light and,

a second part, containing a developer, capable of being applied temporarily onto the photosensitive layer of the first part, to demonstrate any exposure of the photosensitive layer by the exposure light coming from the light conversion layer.

In a storage configuration, the second part is not put into contact with the first part. This means the detector can be kept for a long period before use.

Use of the detector comprises successive steps in which:

at least one portion of the second part is applied onto one surface of the corresponding first part of the photosensitive layer,

the detector is put in the presence of a detection environment,

the light conversion layer is exposed to excitation light, and

at least one surface of the photosensitive layer is freed to visually check its exposure or non-exposure.

Detector operation is as follows. When the detector's environment has not encountered target molecules, e.g. explosives, the printed polymer precursor stays free and does not convert the excitation light into exposure light. The light conversion layer is thus inactive and the exposure layer does not receive exposure light. It is not exposed. The developer of the detector's second part therefore has no effect on the photosensitive layer, and that retains its initial color. It is for example green.

However, when the detector has been in the presence of target molecules, these will have had the opportunity to combine with the printed polymer precursors to form light converters. The light converters are then capable of converting the excitation light into exposure light. The precursors are the MIP molecules already mentioned in the introductory part of the description that are known for the modification of their radiation behavior when they are combined with a target molecule or compound.

The exposure light, supplied by the light converters in response to the excitation light, exposes the photosensitive layer and the developer demonstrates this exposure. The action of the developer is then shown by a color change. In particular this is a darkening when the photosensitive layer is silver-based.

The spectra of the excitation light and exposure light are offset. Preferably, they are completely separate, but can possibly have a slight overlap. Exposure light spectrum means the light spectrum to which the photosensitive layer is sensitive.

In particular, the excitation light can be chosen in the ultraviolet spectrum, and the sensitivity to exposure light can be chosen in the visible or infrared spectra.

The sensitivity spectrum of the exposure layer to the exposure light can be adjusted by a choice of sensitizing dyes in this layer. In this matter one can refer to the document (3) whose references are given at the end of the description. Further, the selective character of the sensitivity to the exposition light, and not to the excitation light, can be caused or at least reinforced by filters.

Indeed, in a particular embodiment of the detector, the photosensitive layer can be in one piece with the light conversion layer by means of a first filter layer preventing the passing of the excitation light. This particular measure enables a certain overlapping of the spectra of the excitation light and the exposure light to be tolerated. The filter enables a partial exposure of the photosensitive layer by the excitation light to be prevented when the detector is subject to the excitation light for a long time.

Further, the light conversion layer has a free surface, opposite the photosensitive layer that can be covered with another filter layer opaque to the exposure light and transparent to the excitation light.

This second filter layer enables, if necessary, the detector to be exposed to the excitation light, by avoiding special precautions to prevent exposure of the photosensitive layer to interference ambient light. This is useful especially when the photosensitive layer is sensitive to visible light.

In the absence of the second filter layer, exposing the detector to the excitation light in an environment protected from visible light and affixing an opaque cover onto the detector after exposure can be envisaged. However these measures are more restrictive.

According to another particular embodiment of the detector, the light conversion layer can have a free surface, opposite the photosensitive layer, the free surface being covered by a protection layer opaque to the exposure light and the excitation light, the protection layer being a peel-off layer. The protection layer can, if necessary, cover the second filter layer.

The purpose of the opaque layer is to protect the photosensitive layer from any light or radiation before use. This means that the detector can be stored in the light. The opaque layer is peel-off for easy removal when using the detector.

One free surface of the photosensitive layer, opposite the light conversion layer can also be covered by a filter layer opaque to the excitation light, and possibly by a protection layer opaque to the excitation and exposure light. These layers again have a protection-before-use role.

One of the first part and the second part of the detector preferably have an adhesive layer that makes the developer in one piece with the photosensitive layer. Using a peel-off adhesive sticker also makes removal of the second part easier to bare the photosensitive layer and to check its possible exposure. However, the photosensitive layer can be bared on the surface opposite the one receiving the developer.

It should be noted that the second part of the detector is not permanently in contact with the photosensitive layer during the storage phase before use. The purpose of this is to prevent a slow but lengthy chemical reaction between the developer and the photosensitive layer. Indeed such a reaction would produce a gradual color change of the photosensitive layer, even if this had not been exposed.

In a particular embodiment of the detector, this can have the general form of a tape. The second part is then in one piece with one surface of the first part, opposite the photosensitive layer, in a storage-before-use configuration, and is capable of being peeled off said surface, to be attached to a photosensitive layer in a use configuration.

Before the assembly of the photosensitive layer and the second part containing the developer, the detector forming a tape can be placed to surround an object, such as, for example, the handle of a case. In particular, a half-turn twist of the strip can be made before applying the second part, or a portion of the second part, onto the photosensitive layer.

The invention also relates to the use of a detector as described in the detection of target compounds taken in a target group comprising the nitrobenzenes, nitrophenols, nitrotoluols, derivatives of atrazine substituted by nitro or hydroxy groups, nitrated derivatives of polyols, as well as bacteria, yeasts or viruses, in their active or dormant state.

Other characteristics and advantages of the invention will appear in the following description, with reference to the figures in the appended drawings. This description is given purely as an illustration and is not limiting.

### BRIEF DESCRIPTION OF THE DRAWINGS

FIG. 1 is a highly enlarged cross-section of a detector according to the invention.

FIG. 2 is a flowchart showing the main steps of a detection method for the presence of hazardous molecules using a detector according to the invention.

FIG. 3 is an illustration of a particular use of a detector compliant with the invention.

### DETAILED DESCRIPTION OF THE INVENTION

In the following description, identical, similar or equivalent parts of the various figures are marked by the same reference signs. Further, it should be noted that the drawings are not shown with a uniform scale, for reasons of clarity of the figures.

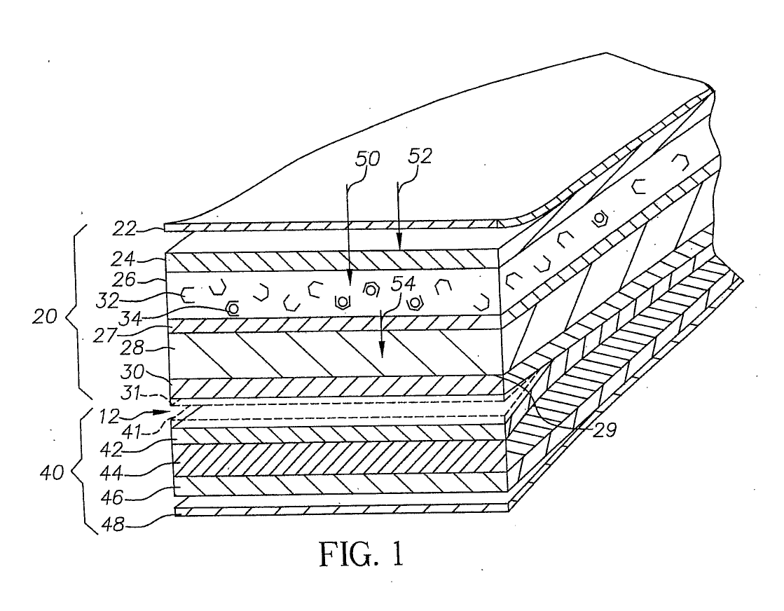

A detector in accordance with the invention can have various forms. It can be for example, in the form of a self-adhesive sticker, in the form of a tape or some other form. Generally it has surfaces whose dimensions are much greater than its thickness. On FIG. 1, the thickness is exaggerated, to better detail the detector's structure.

The detector 10 of FIG. 1 essentially has two parts 20 and 40. It is shown in a use configuration where the two parts are in one piece so as to interact. The interactions between the parts are described below. A slight separation 12 is simply to show more clearly the limit between the two parts 20, 40.

The main function of the first part 20 is to detect the presence of target molecules in the detector's environment. The function of the second part, in interaction with the first part, is to demonstrate the result of the detection visibly for a user.

The first part comprises in order an opaque protection layer 22, a first filter layer 24, a light conversion layer 26 and a photosensitive layer 28. The protection layer 22 is a layer that can be removed by peeling off, which covers the first filter 24. The filter 24 covers the light conversion layer 26, which is itself in contact with the photosensitive layer 28. Finally a second filter layer 30 covers the surface of the photosensitive layer 28 opposite the light conversion layer.

The light conversion layer 26 constitutes the detector's core. It contains imprinted polymer molecules.

In general, they are polymers obtained by free radical polymerization like the polyacrylics, polymethacrylics, polyvinyls and their esters and copolymers, and polyurethane type polymers. Silicon-based inorganic polymers are also suitable.

The polymers are formed from monomers including some giving them properties of recognition of the target compounds and their radiation behavior when they are combined with the target molecules. Such monomers are for example acrylic or methacrylic acids substituted by phenyl halogeno derivatives or the previous acid esters. For illustration refer to the documents given at the end of the description.

At the time of the polymerization, i.e. the manufacture of the MIPs, pore-forming agents can be introduced that do not contribute to the recognition function but increase the porosity and thus the recognition kinetic of the target molecules. These agents are for example hydroxypropylcellulose, or methylcellulose. The MIP molecules can be suited to one or more target molecules capable of being detected. As regards explosives, the target molecules can be nitrobenzenes, nitrophenols, nitrotoluols, atrazine derivatives substituted by nitro or hydroxy groups, or nitrated polyol derivatives. In general, the more the MIP molecule is sterically and electronically close to the system to be detected, the better the selectivity will be. For detecting chemical agents, the target molecules are mainly halogenophosphonic acids. The target compounds can also be bacteria, yeasts, viruses, proteins, or pieces of living organisms. To manufacture MIPs targeting such compounds or systems, refer to document (4), whose reference is given at the end of the description.

The list of MIPs is not exhaustive. Reference can also be made to document (2), mentioned above, which gives examples of MIPs that can be used for detection. As stated above, MIP molecules have the special feature of both being able to combine selectively with the target molecules, and having different radiation behavior in the free state and in the combined state. In other words, MIP molecules can be used as color converters, i.e. as dyes.

When MIP molecules are not combined, like molecule 32 of FIG. 1, they do not modify any excitation light to which they are subject. More precisely, they do not modify the wavelength domain and the maximum wavelength of the excitation light. However, when the MIP molecules are combined with a target molecule to which they correspond, which is the case of molecule 34 in the figure, they are capable of modifying the wavelength domain and the maximum wavelength of the excitation light.

While an implementation of the invention can be envisaged with light converters producing wavelength modification within the visible spectrum, MIP molecules producing a large wavelength modification are preferred. For example, MIP molecules capable of converting ultraviolet excitation light into visible or infrared light when they are combined with a target molecule are selected. The light produced by the MIP molecule in response to the excitation light is still called “exposure light”.

When the protection layer 22 is removed, ultraviolet excitation light 50 supplied by an external lighting device, not illustrated, crosses the first filter layer 24 to reach the conversion layer 26. The filter is, for example, a sheet of Kodak Wratten 39 filter, or a dye or dye mixture in the gel, or a polymer binder having the required extinction domain in the visible spectrum but not in the UV spectrum (ultraviolet). It also lets pass the ultraviolet excitation light, shown by a double arrow, but stops any interference visible light 52, shown by a single arrow.

When the excitation light 50 reaches one of the MIP molecules combined with a target molecule, it is absorbed and then retransmitted with a different wavelength. Here it is retransmitted in the form of visible exposure light.

The exposure light is shown by a single arrow and marked with the reference 54. Its effect is to expose the photosensitive layer 28. The photosensitive layer 28 is, for example, a black and white type photographic layer, with silver halides. It has a dual role. A first role is to make visible later on the fact that the light conversion mechanism and thus the combination of the MIP molecules with the target molecules has taken place in the conversion layer. Another especially important role of the photosensitive layer is an amplification role. Indeed, the number of target molecules present in the environment is usually very low. The number of MIP molecules capable of being combined with the target molecules is thus also low. Therefore the exposure light is of very low intensity. The use of a photographic layer enables a very high amplification factor. Here amplification factor means in the sense that a very low light level is capable of being demonstrated, after development, by a significant color change. The amplification can be reinforced by the sensitization of the silver grains in the photosensitive layer, which has the effect of increasing their sensitivity by a factor of about 10⁹.

An intermediate filter layer 27 is placed between the conversion layer 26 and the photosensitive layer 28. This is a Kodak Wratten 2B or 2E filter layer, or a dye or dye mixture in the gel, or a polymer binder having the required extinction domain in the UV but not in the visible domain. The filter layer lets the exposure light pass while stopping the excitation light. It enables a photosensitive layer 28 to be used with a less selective sensitivity spectrum. The sensitivity spectrum of the photosensitive layer is ideally centered on the spectrum of the exposure light emitted from the conversion layer 26. However, it can be that the sensitivity to the excitation light is not zero. In this case the intermediate filter layer 27 protects the photosensitive layer from an interference exposure of the excitation light.

Similarly, the second filter layer 30 protects the free surface 29 of the photosensitive layer opposite the conversion layer. This is, for example, a dispersion of carbon particles in gelatin or another polymer binder such as polyvinyl alcohol or polyvinylpyrolidone. Its role is to protect the photosensitivity layer when its free surface 29 risks being subject to the excitation light and/or ambient light.

As shown in FIG. 1, the free surface 29 is put into contact, by means of the second filter layer, with the second part 40 of the detector. When this operation takes place in the presence of excitation light only, the layer 30 protects the photosensitive layer from interference exposure.

However, if assembly of the first and second parts of the detector is planned to take place in an environment with visible light, the filter layer 30 is also planned to protect the photosensitive layer from visible light. The layer 30 is then replaced by a filter layer stopping visible light or possibly by an opaque layer. If assembly of the first and second part is planned in the absence of light, and if the second part is itself opaque, the filter layer 30 can possibly be omitted.

Finally, it should be noted that an opaque protection layer 31, shown by a broken line in FIG. 1, can cover the free surface 29 of the photosensitive layer or, if necessary, the filter layer 30, during a storage phase of the detector. This protection layer 31, preferably peel-off, is removed in the use configuration as shown.

The second part of the detector 40 has a developer role. It is mainly formed by a reservoir layer 44 containing a developer. The reservoir layer 44 is, for example, a layer of gelatin or organic polymer such as polyvinyl alcohol or polyvinyl-pyrolidone. It is soaked with or contains a dispersion of conventional photographic developer, blocked or not, of the type used in photography. It is, for example, hydroquinone, derivatives of hydroquinone, dimezone, amino phenol, ascorbic acid, or para-phenylenediamines.

When the first and second parts of the detector are assembled, the reservoir layer is applied against the photosensitive layer 28 or against the filter layer 30 covering it. The filter layer has, if necessary, a porosity enabling the developer to diffuse through to the photosensitive layer.

The exposed parts of the photosensitive layer are revealed by the developer, which causes a color difference between the exposed parts of the photosensitive layer and the unexposed parts of the photosensitive layer. For example an unexposed photosensitive layer can be green and become black following exposure and development.

The second part of the detector 40 is preferably not in permanent contact with the photosensitive layer 28, to prevent a slow chemical reaction between the silver grains and the developer from eventually causing a color change of the photosensitive layer, in the absence of any target molecules.

In order to enable extended storage, the second part of the detector is only put into contact with the photosensitive layer at the time of use. Putting into contact can take place before or after exposure of the detector to the excitation light, but in a sufficiently short time not to cause an interference chemical reaction.

Reference 42 denotes a development activator layer. It is, for example, soda, potash, lithium hydroxide, amines, or any very basic product in a binder. The activator layer 42 covers the reservoir layer and is to be found at the interface between the first and second part of the detector, in its use configuration. A layer 41, shown by a broken line, represents an adhesive layer, possibly peel-off, that enables contact to be maintained between the first and second parts of the detector.

References 46 and 48 denote respectively a reflection layer and an opaque protection layer that cover the free surface of the reservoir layer.

The reflection layer is a semi-transparent layer, made from a material like TiO₂ whose function is to reflect towards the photosensitive layer any excitation light that has crossed the photosensitive layer without interacting with a silver grain. This enables better use of the excitation light produced. The use of the reflection layer 46 is combined with the use of a second filter layer 30 that lets the exposure light through. The semi-transparent character of the reflection filter layer also enables color aging to be checked, i.e. the state of the photosensitive layer when the opaque protection layer 48 is removed.

FIG. 1 represents the detector in a use configuration in which the developer can migrate from the reservoir layer 44 towards the photosensitive layer 28. In a storage configuration, the first and second parts are not in contact, or, at least their contact does not enable developer migration. In a particular embodiment, the second part 40 can have one of its surfaces in contact with the protection layer 22 of the first part 20 in the storage configuration. The peel-off adhesive layer 41 is, for example, stuck to the protection layer 22 of the first part.

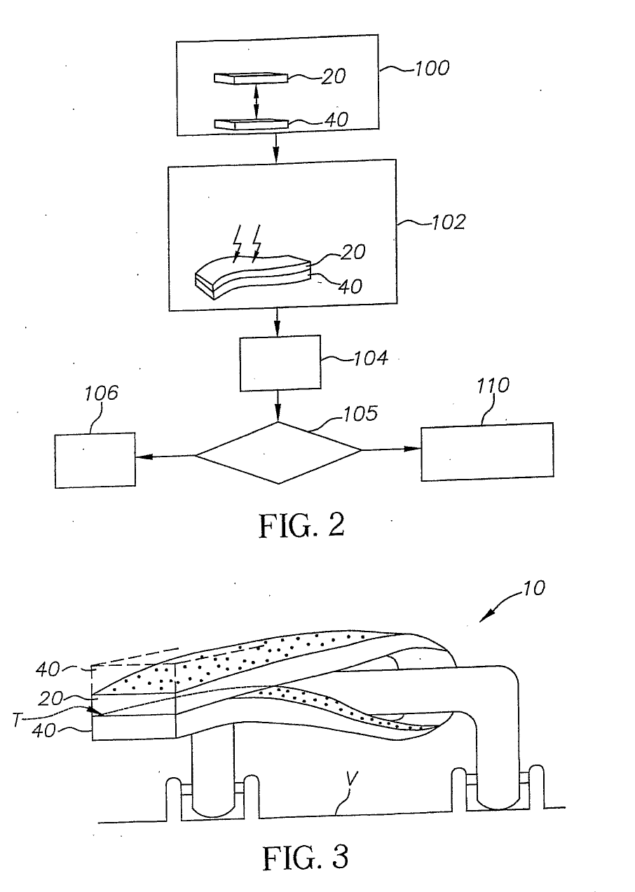

FIG. 2 shows the main steps of a detection method of hazardous molecules in an environment, using a detector according to FIG. 1.

A first step 100 comprises the first and second parts of the detector being put into contact to enable, at least locally, an interaction between the developer and the photosensitive layer described above. This first step can also comprise the detector being put into the environment to be inspected. This is, for example, the affixing of the detector onto an object to be inspected, such as a case.

A second step 102 comprises the exposure of the detector to an excitation light. The role of the excitation light, already explained, is not repeated here.

A third step 104 consists in freeing the photosensitive layer to visually report its color change or on the contrary report an absence of color change. Freeing the photosensitive layer means either its baring, by detaching the second part of the detector from the first part, or the removal of an opaque cover enabling the color of the layer to be seen through one or more other layers. With reference to FIG. 1, it is possible to detach, for example, the second part by the start of separation 12. When the filter layer 30 of the first part is not transparent to visible light it is also possible to remove this layer.

According to another possibility, provided when the reflection layer 46 and the reservoir layer are sufficiently transparent to let the color of the photosensitive layer be seen, the protection layer 48 has just to be removed from the second part 40. The use of peel-off adhesive stickers facilitates the “freeing” of the photosensitive layer.

The order of certain steps of the method is free. For example the detector can be subjected to an environment to be inspected before or after the detector having been put into its use configuration. However, it is useful to proceed to the visual check of the color change of the photosensitive layer rapidly after having freed this layer. Freeing the photosensitive layer enables its color to be checked visually, but also has the effect of submitting it to the ambient light, and thus rapidly causing its exposure. Part of the developer that has diffused into the photosensitive layer indeed causes a color change, in this case a gradual darkening of the layer in the presence of light. The darkening can take place in a few seconds. The visual check can be made possibly under controlled light to prevent any action of the ambient light and extend the time of the visual check.

In general, the visual check must be carried out before the color change of the photosensitive layer in response to the ambient light.

A lozenge 105 indicates a choice depending on the color of the photosensitive layer at the moment of its freeing. Either the color remains unchanged, green for example. In this case the object bearing the detector does not contain hazardous material. The object is considered as risk free and can be loaded on a plane. This action is shown with the reference 106.

However, if the color of the photosensitive layer is found modified, locally or fully, at the moment of its freeing, one may conclude that the object bearing the detector has encountered the hazardous material. This does not necessarily mean that the object, for example the case, contains the hazardous material or explosives, but simply that the target molecules corresponding to this material have been detected. The object or case is thus considered as suspect and must undergo a thorough inspection before deciding if it can be embarked or not. This action is shown with the reference 110.

FIG. 3 shows a particular use of a detector 10 according to the invention in which the second part 40 is found sometimes on one side of the first part 20 in a storage configuration and sometimes on the opposite side in the use configuration. The storage configuration is drawn in broken lines.

The special feature of the detector of FIG. 3 is having the form of a tape 10 and being usable as a baggage check-in label in an airport. The tape is affixed at the time of check-in. One end of the tape that constitutes the second part 40 of the detector is detached from the first part 20 and is passed round the handle of a case V before being stuck back onto the opposite surface of the first part. According to the order of the layers forming the first and second parts of the detector, the tape 10 is twisted like a Möbius strip, so as to put the reservoir layer into contact with the photosensitive layer.

The case and tape are then subjected to strong ultraviolet lighting during their transfer towards the baggage loading station.

The baggage handler then frees one part of the photosensitive layer of the detector and visually checks in a test region T if a color change has taken place or not.

The detector of FIG. 3 can be formed by initially laying the various layers onto a support layer, which is, for example, the protection layer 22 mentioned above. In this case, the first and second parts of the detector would be found on the surfaces opposite this layer in the storage configuration.

### REFERENCE DOCUMENTS

(1) US 2003/0082635 A1
(2) US 2003/0027936 A1
(3) The theory of photographic process 4th edition 1977—T. H. James—chapters 8, 9, 10
(4) F. L. Dickert, O. Hayden (2002) Anal. Chem, 74, 1302-1306 and Mat. Res. Soc. Symp. Proc. Vol 723, 2002, 25
(5) WO 03/012410

1) A single-use detector of target compounds comprising:
a first part (20) including a light conversion layer (26), and a photosensitive layer (28) in one piece with the light conversion layer, to be capable of being exposed by exposure light (54) capable of being produced by the light conversion layer in response to excitation light (50), the light conversion layer containing an imprinted polymer precursor (32), capable of combining with a target compound to form a dye (34) that converts the excitation light into exposure light in a wavelength domain different from that of the exposure light and,
a second part (40) of developer, capable of being applied temporarily to the photosensitive layer (28) of the first part (20) to demonstrate a possible exposure of the photosensitive layer.

2) A detector according to claim 1, wherein the photosensitive layer (28) is in one piece with the light conversion layer (26) by means of an intermediate filter layer (27) preventing the passing of the excitation light.

3) A detector according to claim 1, wherein the light conversion layer (26) has a free surface, opposite the photosensitive layer, the free surface being covered with another filter layer (24) opaque to the exposure light and transparent to the excitation light.

4) A detector according to claim 1, wherein the light conversion layer (26) has a free surface, opposite the photosensitive layer, the free surface being covered with a second protection layer (22) opaque to the exposure light and to the excitation light, the protection layer (22) being a peel-off layer.

5) A detector according to claim 1, wherein the photosensitive layer (28) has a free surface (29) opposite the light conversion layer (26), the free surface (29) being covered with a filter layer (30) opaque to the excitation light.

6) A detector according to claim 1, wherein the photosensitive layer (28) has a free surface (29), opposite the light conversion layer (26), the free surface being covered with a peel-off protection layer (31) opaque to the excitation light and to the exposure light.

7) A detector according to claim 1, wherein the excitation light is light in the ultraviolet spectrum, and wherein the exposure light is light in the visible or infrared spectrum.

8) A device according to claim 1, wherein the second developer part (40) comprises a reservoir layer (44) containing a developer capable of diffusing from the reservoir layer towards the photosensitive layer (28) when the developer part is applied to the photosensitive layer.

9) A device according to claim 8, wherein the second part (40) also comprises an activator layer (42) covering one surface of the reservoir layer (44) capable of being applied to the first part (20) of the detector.

10) A detector according to claim 9, wherein the reservoir layer (44) has a free surface covered by at least one of a reflection layer (46) and an opaque protection layer (48).

11) A detector according to claim 1, wherein at least one of the first and second parts has an adhesive peel-off layer (41) to make the second part in one piece with the photosensitive layer.

12) A detector according to claim 2, wherein the second part (40) is in one piece with a surface of the first part (20), opposite the photosensitive layer, in a storage-before-use configuration, and is capable of being peeled off said surface, to be attached to the photosensitive layer (28) in a detection configuration.

13) A detector according to claim 1 in strip form, comprising a support layer (22), wherein the first and second parts (20, 40) are arranged on the surfaces opposite the support layer, in a storage-before-use configuration.

14) A use of a detector according to claim 1, for the detection of target compounds taken in a target group comprising the nitrobenzenes, nitrophenols, nitrotoluols, derivatives of atrazine substituted by nitro or hydroxy groups, nitrated derivatives of polyols, bacteria, yeasts or viruses.

15) A method of detection of target molecules using a detector according to claim 1, comprising:
applying at least one portion of the second part (40) onto the photosensitive layer of the first part (40),
putting the detector in the presence of a detection environment,
exposing the light conversion layer to excitation light (50), and
freeing at least one surface of the photosensitive layer (28) to visually check its exposure or non-exposure.

16) A method according to claim 15, wherein a detector is used in strip form, comprising a support layer (22), the first and second parts being arranged on the surfaces opposite the support layer (22), in a storage-before-use configuration, and wherein a half-turn twist of the strip is made before applying a portion of the second part to the photosensitive layer.
